(19) United States
(12) Patent Application Publication
(10) Pub. No.: US 2025/0205379 A1
(43) Pub. Date: Jun. 26, 2025

(54) MICRO-LEDS CONFIGURED FOR OPERATION AT WAVELENGTHS IN THE FAR-UVC SPECTRUM

(71) Applicant: Uviquity, Inc., Raleigh, NC (US)

(72) Inventors: Brent Fisher, Bethesda, MD (US); Scott Burroughs, Raleigh, NC (US)

(21) Appl. No.: 18/844,525

(22) PCT Filed: Mar. 15, 2023
(86) PCT No.: PCT/US2023/015268

§ 371 Date: Sep. 6, 2024

Related U.S. Application Data
(60) Provisional application No. 63/320,379, filed on Mar. 16, 2022.

(51) Int. Cl. A61L 2/10; A61L 9/20; H10H 20/855; H10H 20/857

(52) U.S. Cl. CPC A61L 2/10; A61L 9/20; H10H 20/855; H10H 20/857; A61L 2202/11; A61L 2209/12

(57) ABSTRACT

A light emitting diode (LED) includes a semiconductor structure comprising at least one epitaxial layer that is configured to generate far-UVC light, and respective electrical contacts on first and second surfaces of the semiconductor structure. One or more dimensions of the at least one epitaxial layer in a lateral direction are within an order of magnitude of a thickness of the at least one epitaxial layer in a vertical direction. A primary light extraction surface may include at least one sidewall of the semiconductor structure that extends between the first and second surfaces and is configured to emit the far-UVC light. Related devices, arrays thereof, and fabrication methods are also discussed.

### CLAIM OF PRIORITY

The present application claims priority from U.S. Provisional Patent Application No. 63/320,379 filed Mar. 16, 2022, with the United States Patent and Trademark Office, the disclosure of which is incorporated by reference herein in its entirety.

### FIELD

The present application is directed to UV light sources, and in particular, to far-UVC light sources and related devices and methods.

### BACKGROUND

Compact and efficient ultraviolet (UV) light sources in the wavelength range of about 200 nm to about 400 nm may be desirable for many applications. For example, there is an emerging market for germicidal UV (GUV) products. UVC light including photons in the UVC wavelength range (e.g., with wavelengths of about 200 nm to about 280 nm; also referred to as UV-C) can be used to disinfect airborne and surface disease-causing pathogens, while remaining safe for human exposure. With respect to human safety, far-UVC light (with wavelengths from about 200 nm to about 240 nm) may not penetrate through the dead-cell layer of the skin surface or the tear layer of the human eye. In particular, far-UVC light can efficiently cause permanent physical damage to DNA and/or proteins, which can prevent bacteria, viruses and fungi from replicating. Human-safe far-UVC light can thus effectively kill or inactivate disease causing pathogens with little to no risk to humans because these wavelengths may be largely absorbed by the stratum corneum (the top layer of dead skin cells in the epidermis) or tear layer of the eye. That is, light in the far-UVC wavelength range may be capable of rapidly killing microscopic pathogens (like bacteria) and inactivating viruses, yet cannot penetrate human skin deep enough to pose threat of harm to humans. This may allow for disinfecting air and surfaces using UV light in the presence of humans.

However, operation in the far-UVC wavelength range may present challenges. For example, few available light sources may be configured for operation in the far-UV. Some conventional far-UVC light sources have been implemented by gas-discharge lamps, which generate light from excimers formed in the transient plasma. For example, KrCl excimer lamps may generate light at 222 nm. Solid state sources of far-UVC light (e.g., light emitting diodes (LEDs), in some instances using phosphor-based wavelength conversion) may be desirable for reducing cost and/or increasing reliability, among other benefits. While LEDs may be widely deployed and developed solid state light sources, such light sources typically have short operating lifetimes and poor performance at emission wavelengths shorter than about 265 nm. Thus, LEDs that emit in the far-UVC wavelength range may not be commercially available.

### SUMMARY

According to some embodiments, a light emitting diode (LED) includes a semiconductor structure comprising at least one epitaxial layer that is configured to generate far-UVC light. One or more dimensions of the at least one epitaxial layer in a lateral direction are within an order of magnitude of a thickness of the at least one epitaxial layer in a vertical direction.

In some embodiments, the semiconductor structure includes first and second surfaces having respective electrical contacts thereon, and at least one sidewall that extends between the first and second surfaces and is configured to emit the far-UVC light.

In some embodiments, the at least one sidewall comprises opposing sidewalls of the at least one epitaxial layer that extend between the first and second surfaces, and the one or more dimensions comprise a distance between the opposing sidewalls.

In some embodiments, the at least one sidewall is configured to direct the far-UVC light into a beam or into a distributed pattern.

In some embodiments, the at least one sidewall is inclined between the first and second surfaces.

In some embodiments, at least one of the respective electrical contacts is at least partially opaque to the far-UVC light.

In some embodiments, a substrate includes the semiconductor structure on a front surface thereof. The substrate is different than a native substrate on which the semiconductor structure is formed.

In some embodiments, the substrate comprises one or more optical redirection structures facing the at least one sidewall of the semiconductor structure and configured to alter a propagation direction of the far-UVC light emitted therefrom into one or more directions.

In some embodiments, the one or more optical redirection structures are attached to the front surface of the substrate adjacent the semiconductor structure.

In some embodiments, the one or more optical redirection structures are integral to the substrate, and the surface of the substrate including the semiconductor structure thereon is recessed relative to the one or more optical redirection structures.

In some embodiments, the substrate comprises a back surface opposite the front surface and having a backside contact thereon, and at least one conductive through via that extends through the substrate and electrically connects at least one of the respective electrical contacts of the semiconductor structure to the backside contact.

In some embodiments, the LED is free of a native substrate of the semiconductor structure.

According to some embodiments, a light emitting diode (LED) includes a semiconductor structure that is configured to generate far-UVC light, the semiconductor structure comprising first and second surfaces, respective electrical contacts on at least one of the first and second surfaces, and a primary light extraction surface comprising at least one sidewall of the semiconductor structure that extends between the first and second surfaces and is configured to emit the far-UVC light.

In some embodiments, the at least one sidewall comprises opposing sidewalls of one or more epitaxial layers of the semiconductor structure that extend between the first and second surfaces, and a distance between the opposing sidewalls is less than about 100 microns.

In some embodiments, the distance between the opposing sidewalls is less than about 50 μm.

In some embodiments, a thickness of the one or more epitaxial layers of the semiconductor structure is about 10 μm or less.

In some embodiments, the distance between the opposing sidewalls is within an order of magnitude of the thickness of the one or more epitaxial layers of the semiconductor structure.

In some embodiments, the at least one sidewall is inclined between the first and second surfaces.

In some embodiments, at least one of the respective electrical contacts is at least partially opaque to the far-UVC light.

In some embodiments, at least a portion of the first and/or second surfaces comprise light extraction surfaces that are configured to emit the far-UVC light.

According to some embodiments, a light emitting diode (LED) includes a semiconductor structure that is configured to generate light comprising a wavelength in a far-UVC wavelength range. The semiconductor structure includes first and second surfaces having respective electrical contacts thereon and one or more lateral surfaces extending between the first and second surfaces. A collective surface area of the one or more lateral surfaces is within a factor of 10 of a respective surface area of the top or bottom surface.

According to some embodiments, a light emitting diode (LED) array includes a common support substrate, and a plurality of the LEDs as described herein, arranged on a surface of the common support substrate. Optical redirection structures may be provided on the surface of the common support substrate between adjacent ones of the plurality of the LEDs.

In some embodiments, the semiconductor structure of any of the LEDs described herein is further configured to generate UVC light.

Other devices, apparatus, and/or methods according to some embodiments will become apparent to one with skill in the art upon review of the following drawings and detailed description. It is intended that all such additional embodiments, in addition to any and all combinations of the above embodiments, be included within this description, be within the scope of the invention, and be protected by the accompanying claims.

### BRIEF DESCRIPTION OF THE DRAWINGS

FIGS. 1A and 1B are schematic perspective and plan views, respectively, illustrating configurations of a conventional macroLED light source that provides light emission in the far-UVC spectrum.

FIGS. 2A and 2B are schematic perspective and plan views, respectively, illustrating configurations of a microLED light source that provides far-UVC light generation according to some embodiments of the present disclosure.

FIGS. 3A and 3B are schematic cross-sectional views illustrating configurations of a microLED light source including sidewalls that are configured to direct the far-UVC light in one or more desired directions according to some embodiments of the present disclosure.

FIGS. 4A and 4B are schematic cross-sectional views illustrating configurations of a microLED light source on a substrate including optical redirection structures that are configured to direct the far-UVC light in one or more desired directions according to some embodiments of the present disclosure.

FIGS. 5A and 5B are schematic cross-sectional views illustrating configurations of a microLED light source on a substrate including recessed surfaces and sidewalls that are configured to direct the far-UVC light in one or more desired directions according to some embodiments of the present disclosure.

FIGS. 6A and 6B are schematic cross-sectional views illustrating configurations of a microLED light source on a substrate including recessed surfaces, sidewalls that are configured to direct the far-UVC light in one or more desired directions, and through-via electrical connections extending therethrough according to some embodiments of the present disclosure.

FIGS. 7A and 7B are schematic side views illustrating methods of fabricating a microLED light source on a non-native substrate according to some embodiments of the present disclosure.

### DETAILED DESCRIPTION OF EMBODIMENTS

Embodiments of the present disclosure provide devices that generate electromagnetic radiation in the far-UVC wavelength range (about 200 nm to 240 nm, for example, about 207 nm to 222 nm), which can be useful for numerous applications, including (but not limited to) germicidal applications for disinfecting airborne and surface disease-causing pathogens, and detection of trace chemical or biological species in various field environments (air, water, etc.), while simultaneously remaining safe for human exposure and complying with human safety regulations and requirements. As used herein, “far-UVC” or “Far UVC” wavelength band or range refers to wavelengths greater than about 200 nm (such that the radiation is non-ionizing in the atmosphere), and less than about 240 nm, for example, about 200 nm to 230 nm.

In particular, embodiments of the present disclosure provide configurations for enhancing the performance (i.e., optical output) of inorganic LEDs that emit light in the far-UVC portion of the electromagnetic spectrum, specifically at wavelengths less than about 240 nm. While some custom designed LEDs and research labs have demonstrated LEDs with wavelengths from 260 down to near 230 nm, the efficiency of such devices falls rapidly and thus may not be commercially viable.

Several challenges may hold back development of LEDs configured to emit light at wavelengths below about 250 nm, many of which may be related to extracting light from the device itself. Indeed, the external quantum efficiency (EQE) of UVC LEDs may remain lower than 10% due to difficulties of extracting light.

One such challenge may relate to the top and bottom electrical contacts. In particular, the contact to the p-side of the LED p-n junction (also referred to as the p-contact) may be difficult to form with optical transparency for light of shorter wavelength ranges (e.g., at about 200 nm to 230 nm or 240 nm) as the electrical doping level is increased. That is, while higher doping may be needed to reduce resistance (and increase efficiency), the higher doping levels may reduce optical transparency, which may make it more difficult for photons to exit the LED because of absorption by the electrical contacts. Efficiency may thereby suffer due to the non-transparency of the contacts. Embodiments described herein recognize tradeoffs, including design changes, that may relieve the need for either high doping levels or extracting photons through the surface can benefit the efficiency of the LED.

Another challenge with reducing the wavelength may relate to the directionality of light that is generated inside of the LED. For example, as the aluminum content of the AlGaN material increases, the band gap may increase (and the emission wavelength of the generated light may decrease). Accompanying these changes may be a departure from isotropic generation of photons toward a situation in which the optical transition dipole inside the material may be more and more vertical (relative to the crystal plane of the semiconductor material). The closer the transition dipole approaches an ideal vertical dipole, the more anisotropic the generation of photons. Transverse electric (TE) polarization may be dominant in InGaN/GaN based blue LEDs, whereas transverse magnetic (TM) polarization may be dominant in AlGaN/GaN UV LEDs. More plainly, as the emission wavelength is reduced, the light that is generated inside of the LED may predominantly propagate (i.e., with greater intensity) at lateral angles relative to a plane of the active region (i.e., the quantum well(s)) of the semiconductor material. The light propagating at lateral angles (i.e., within a critical angle for total internal reflection (TIR)) is more likely to be waveguided to the edge and less likely to be emitted out the top or bottom surface of the device. As used herein, propagation may refer to the direction of travel of light (or photons) whether within or outside the semiconductor material, while emission may refer to the output of the light (or photons) from the semiconductor material. Further confounding performance, the length scale for a photon to reach the edge of a macroscopic LED may be thousands of times larger than the distance for the photon to reach the top or bottom surface.

Embodiments of the present disclosure may address the above and other problems by (i) removing constraints on optical transparency or light absorption for the top or bottom (e.g., p-) electrical contact, which can simplify the design, increase the internal quantum efficiency (IQE), and therefore increase the overall efficiency, and (ii) addressing polarization problems that may arise for shorter wavelengths of light (in particular, wavelengths less than about 240 nm, such as far-UVC light) by shortening the optical path to the light extraction surface, taking advantage of the fact that the shorter wavelength light predominantly propagates at lateral angles through the semiconductor material as Al composition increases in the AlGaN multiple-quantum wells for wavelengths less than about 240 nm, which can significantly increase the EQE. This challenge may be referred to as Light Extraction Efficiency (LEE), where EQE=IQE×LEE. Embodiments of the present disclosure may thereby improve IQE and LEE, leading to an overall improvement in EQE.

Embodiments of the present disclosure as described herein relate to the use of microscale dimensions for each individual LED (which may be referred to herein as microLEDs or μLEDs) in order to realize performance enhancements over state of the art macroscale LED designs (which may be referred to herein as macroscopic LEDs or macroLEDs). Benefit may be derived from geometry: by fabricating the LEDs with microscopic lateral dimensions (i.e., within one or two orders of magnitude of the microscopic thickness dimension thereof), the relative contribution of the edge of the LED can be much higher. Further, the average contact resistance can be reduced because of the shorter length scales. Embodiments described herein may be particularly suited for UVC or far-UVC LEDs based on AlGaN semiconductor materials by way of example, but are not limited to AlGaN materials, and may be implemented by other materials as well. Such materials may include (but are not limited to) boron nitride (BN), gallium nitride (GaN), silicon carbide (SiC), aluminum nitride (AIN), or other Group-III nitride or solid state semiconductor material configured to provide light emission in the far-UVC spectrum.

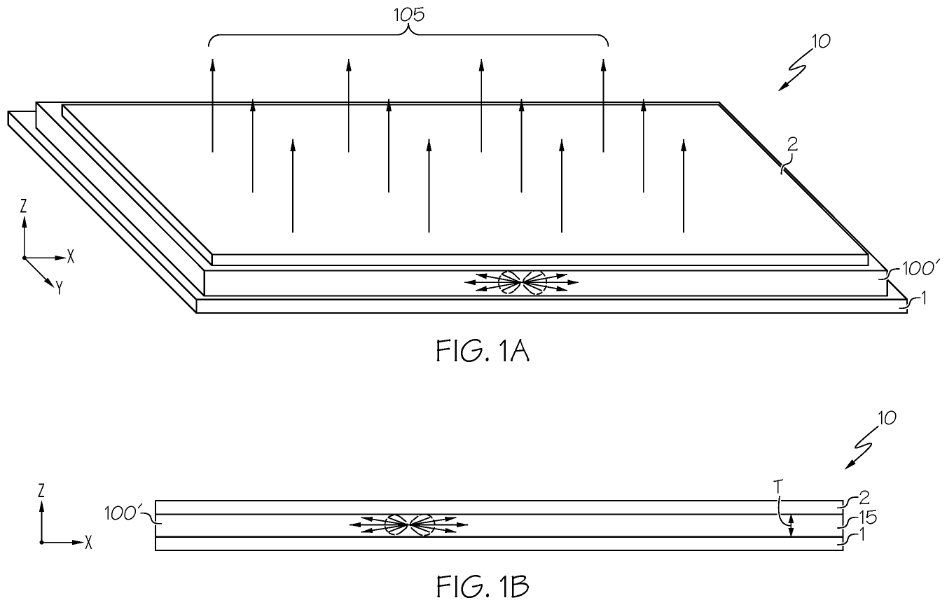

FIGS. 1A and 1B are perspective and plan views, respectively, illustrating configurations of a conventional LED light source that provides light emission 105 in the far-UVC spectrum. In particular, FIGS. 1A and 1B illustrate a conventional macroscopic LED 10 including a semiconductor material 100′ (e.g., an AlGaN layer) with respective electrical contacts 1, 2 (e.g., an n-contact and a p-contact) on top and bottom surfaces of the semiconductor material 100′. The macroscopic LED 10 has a length dimension (e.g., along the x- or y-axis) that is larger (e.g., tens or thousands of times greater) than the thickness T of the macroscopic LED 10. For example, the thickness T may be about 50 μm or less, while the length dimension (e.g., along the x-direction) may be about 1 millimeter (mm) or more. Macroscopic LEDs 10 are thus typically configured to couple the light out the top (or bottom) surface as a light extraction surface. However, such outcoupling may be challenging for light in the far-UVC spectrum, due for example to difficulties relating to the directionality of the far-UVC light 105 that is generated in the LED semiconductor material 100′ (e.g., with greater intensity along the horizontal or lateral direction), and the degree or amount of transparency of the electrical contacts 1, 2 to the far-UVC light 105. In particular, as noted above, it may be difficult to form the p-contact 1 with both the desired electrical characteristics (e.g., low resistance) and the desired optical characteristics (e.g., transparency to light in the far-UVC spectrum).

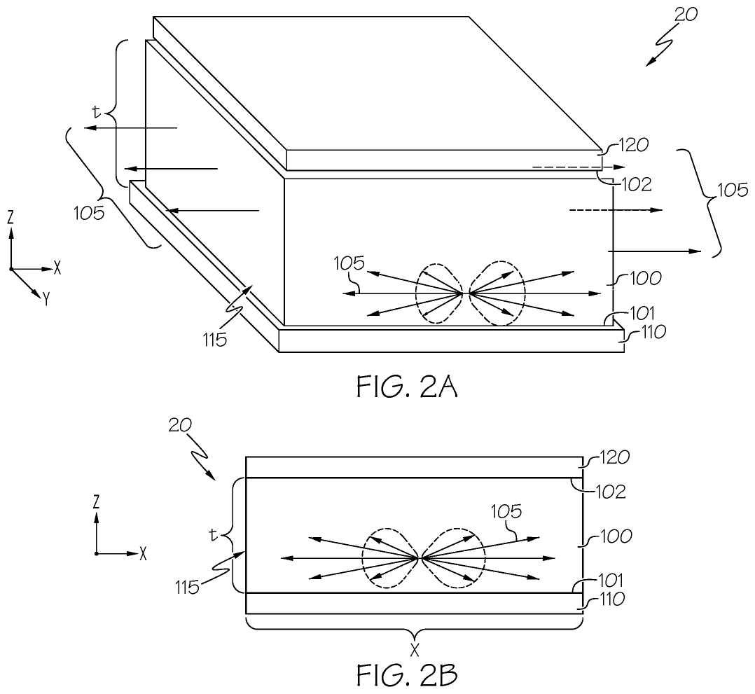

Embodiments of the present disclosure provide “micro” LEDs in which the lateral length scale (e.g., in the x-and/or y-directions) of the LED is reduced, which may address and/or obviate the above and/or other challenges. FIGS. 2A and 2B are perspective and plan views, respectively, illustrating configurations of a microLED light source 20 that provides far-UVC light 105 generation according to some embodiments of the present disclosure. While described primarily with reference to generation of far-UVC light, the light output 105 of the microLED light sources is not limited to the far-UVC wavelength range, and in some embodiments may include or may be configured to generate light including wavelengths in the broader UVC wavelength range (also referred to herein as UVC light).

FIGS. 2A and 2B illustrate a microLED 20 in accordance with some embodiments of the present disclosure, including differences as compared to the macroLED 10 as shown in FIGS. 1A and 1B. In both cases, the thickness of the LED 10, 20 may be determined by the thickness T, t of the semiconductor structure 100′, 100. In a microLED 20 in accordance with some embodiments of the present disclosure, the thickness t may be in the range of about 0.1 micrometer (um) to about 20 μm, for example, about 0.1 μm to about 10 μm, or about 0.5 μm to about 5 μm. As used herein, a semiconductor structure 100 may include one or more semiconductor material layers configured to generate light in the far-UVC wavelength range, also referred to as far-UVC light 105. The semiconductor material may be formed (e.g., epitaxially grown) on a source substrate or wafer (referred to herein as a native substrate), which may or may not be removed from the LED light sources described herein in some embodiments.

For a macroscopic LED 10 as shown in FIGS. 1A and 1B, the lateral dimensions (e.g., along the x-and/or y-directions) of the epitaxial layer can exceed the thickness T by a ratio exceeding about 1000:1. This can present challenges for photons that are emitted in-plane because the LED material typically can re-absorb photons that are emitted. The probability of a photon reaching an edge or sidewall 15 of the semiconductor structure 100′ may fall exponentially with (a) the distance to the edge and (b) the relative strength of the transition dipole in this orientation. Because of this issue, it macroscopic LEDs 10 may include features in the top and/or bottom surfaces 1, 2 to enhance the coupling of light out of the LED through these surfaces. Doing so may increase the extraction efficiency of light from the LED (where photonic extraction efficiency =EQE/IQE).

In contrast to the macroscopic LED 10 shown in FIGS. 1A and 1B, a microLED 20 according to some embodiments of the present disclosure may have semiconductor light emitting layer(s) (e.g., epitaxial layers) with one or more lateral dimensions (e.g., in the horizontal (x- and/or y-) direction(s) or along the surface of the contacts 110, 120 in the figures, also referred to as a lateral direction) that are at approximately the same scale as the thickness of the semiconductor light emitting layer(s) of the device (e.g., in the vertical (z-) direction in the figures, which is perpendicular to the lateral direction(s)). The lateral dimensions may refer to length, width, diameter, long axis, etc., depending on the shape defined by the semiconductor light emitting layers. That is, a microLED as described herein may have epitaxial layer(s) with one or more dimensions x, y in a lateral direction that are on the order of (e.g., having a similar or within at least one order of magnitude or a couple of orders of magnitude of, including less than, greater than, or substantially equal to) a thickness dimension t thereof in the vertical direction. For example, a microLED may include a semiconductor structure 100 having semiconductor epitaxial layer(s) with a collective thickness, t, of about 15 or 20 μm or less (e.g., about 0.1 to 10 μm, or about 0.5 μm to about 5 μm), and may include one or more lateral dimensions x, y of less than about 500 μm (e.g., less than about 100 μm, less than about 50 μm, or about 1 to 10 μm). In some embodiments, the epitaxial layer(s) of the semiconductor structure 100 may have a thickness, t, of about 7 μm to about 12 μm (after removal from a native substrate, for example, via laser lift-off) and may have lateral dimensions x, y between about 3 μm to about 50 μm. The edge(s) or sidewall(s) 115 (also referred to as lateral surface(s)) of the semiconductor structure 100 of the microLED 20 may provide a primary light extraction surface 115.

Benefits of such lateral dimension(s) may arise from recognition that the physical distance that any given photon must traverse before reaching an edge or sidewall of the device to be outcoupled may be orders of magnitude shorter than some macroscopic designs. As shown in FIGS. 2A and 2B, the semiconductor structure 100 is configured to generate photons in the far-UVC wavelength range, which predominantly propagate at substantially lateral angles relative to the active region of the semiconductor material (shown along the horizontal or lateral direction in the figures). As shown by the dashed lines in FIGS. 2A and 2B, intensity of the generated far-UVC light 105 is significantly reduced with deviation from the lateral direction. The lateral dimension(s) of the semiconductor active layer(s) of the microLED 20 are configured to shorten the optical path length for output of the photons (i.e., the far-UVC light 105) at the light extraction surface 115 at one or more sidewalls 115 of the semiconductor structure 100. The reduced physical distance or optical path length of microLEDs 20 including epitaxial layers having lateral dimensions x, y according to some embodiments of the present disclosure may provide an exponentially higher probability of photon extraction at far-UVC wavelengths, and thus, may be critical to improving quantum efficiency to levels sufficient for viability.

More particularly, FIGS. 2A and 2B illustrate a semiconductor structure 100 including a first surface 101, a second surface 102, and respective electrical contacts 110, 120 (e.g., an n-contact and a p-contact) on the first and second surfaces 101, 102. At least one sidewall 115 extends between the first and second surfaces 101, 102 and provides a primary light extraction surface 115 that is configured to emit the far-UVC light 105. In some embodiments, multiple (e.g., opposing) sidewalls 115 of the epitaxial layer(s) of the semiconductor structure 100 may provide the primary light extraction surface 115, and a distance between the opposing sidewalls 115 along the lateral direction may be on the order of (e.g., within one or two orders of magnitude of) the thickness, t, of the epitaxial layer(s) of the semiconductor structure 100 along the vertical direction. For example, the distance between the opposing sidewalls 115 along the lateral direction may be less than about 100 microns (e.g., less than about 50 μm, or about 1 to 10 μm). MicroLEDs 20 including sidewalls or lateral surfaces 115 for primary light extraction may thus be considered as edge-emitting devices.

It will be understood that, while described herein with reference to the sidewalls or lateral surfaces 115 of the semiconductor structure 100 as a primary light extraction surface 115, embodiments of the present disclosure are not limited to light extraction from only the sidewalls or lateral surfaces 115. For example, in some embodiments, at least a portion of the first and/or second surfaces 101, 102 having the respective electrical contacts 110, 120 thereon may also provide light extraction surfaces that are configured to emit the far-UVC light 105, and thus may provide secondary light extraction surfaces.

Benefits of such lateral dimension(s) may also arise from recognition that the relative amount of surface area that is contributed by the lateral edges of a microLED (e.g., on opposing ends of the microLED, in comparison to the surface area of the top and/or bottom surfaces) may be far higher than for a macro LED (where the top/bottom surfaces have substantially greater surface area than the lateral edges). For example, a microLED having lateral dimensions of about 1 μm×1 μm to about 50 μm×50 μm may have top or bottom surface area of about 1 to about 2500 μm², and lateral surfaces 115 with a collective area of about 0.1 to about 2000 μm². As such, in some embodiments, a collective surface area of one or more lateral surfaces 115 of the semiconductor structure 100 may be within a factor of 10 of (e.g., within about 5 times of, or within about 5 times to 10 times of) a respective surface area of the top or bottom surface of the semiconductor structure 100. That is, microLEDs as described herein may include epitaxial layers with one or more lateral dimensions x, y that are on the order of (e.g., within an order of magnitude or within a factor of 5 of) the thickness dimension t thereof, and/or lateral edge surface areas that are on the order of the top/bottom surface areas thereof.

Moreover, by increasing the contribution of the lateral edge surfaces of the device for light emission, microLEDs according to some embodiments of the present disclosure may decouple the photon extraction surfaces from the surfaces upon which electrical current is introduced (typically the top and bottom surfaces 101, 102 having the electrical contacts 110, 120 thereon). As such, problems of and tradeoffs between optical transparency (for efficiency) and doping levels (i.e., contact resistance) can be avoided altogether, thereby providing improved efficiency in the far-UVC spectrum. In some embodiments, one or more of the electrical contacts 110, 120 may be formed of materials and/or doping levels without optical transparency requirements, to provide reduced electrical resistance and/or ohmic contacts to the semiconductor structure 100. In particular, one or more of the electrical contacts 110, 120 may be partially (e.g., greater than 10%, greater than 25%, or greater than 30%) or substantially (e.g., greater than 50%, greater than 75%, or greater than 90%) or completely (100%) opaque to far-UVC light 105. That is, microLEDs including lateral light extraction surfaces 115 as described herein may allow for the use of (semi-) opaque electrical contacts 110 and/or 120, so as to achieve both greater light extraction efficiency and reduced electrical resistance.

For example, for a semiconductor contact, the electrical resistance may include the Contact Resistance (Ohm cm²)/Area (cm²). By reducing the lateral dimensions x, y of the LEDs as described herein, the Area (cm²) can be reduced by orders of magnitude, which may increase the contact resistance. While this may present challenges, microLEDs as described herein may provide primary light extraction surfaces at one or more sidewalls 115 of the device, which may allow for electrical contacts that are not constrained by optical transparency requirements (e.g., the electrical contacts may be partially or completely opaque with respect to the far-UVC light), which may allow for resistance to be reduced and may improve efficiency further still at any doping level. Some embodiments may provide far-UVC emitting LEDs with a contact resistance of less than (e.g., more than a factor of two less than, or more than an order of magnitude less than) that which may be achievable in some macroLEDs (due to the optical transparency requirements for the electrical contacts of the macroLEDs for light emission from the top and/or bottom surfaces). For example, far-UVC emitting LEDs including electrical contacts 110, 120 that are substantially or completely opaque with respect to far-UVC light as described herein may have a contact resistance of less than about 3×10⁻³ ohms/cm² in some embodiments.

FIGS. 2A and 2B illustrate general embodiments of the present disclosure, and in particular, the inventive concepts of the shorter lengths/lateral dimensions (e.g., where the epitaxial layer(s) of the microLED has one or more lateral dimensions x, y of less than 500 μm (e.g., less than about 100 μm, less than about 50 μm, or about 1 to 10 μm) or on the order of the thickness t thereof). Additional example embodiments may include (but are not limited to) one or more of: providing the p-contact on the bottom surface of the microLED (instead of on the top surface) and the n-contact on the top surface (instead of on the bottom surface); patterning the p- and n-contacts on the top and bottom surfaces of the microLED (e.g., on less than an entirety of the surface area of the top and bottom surfaces of the epitaxial layer(s) of the semiconductor structure 100), rather than forming the p-and n-contacts by blanket coverage of the top and bottom surfaces of the semiconductor structure 100; forming the epitaxial layer(s) of the semiconductor structure 100 from materials such as hexagonal boron nitride or gallium nitride (GaN) or silicon carbide (SiC) or pure aluminum nitride (AIN) or other Group-III nitride or solid state semiconductor material configured to provide light emission in the far UVC spectrum; providing the at least one lateral dimension of up to about 100 μm; and/or removal of the native growth substrate of the semiconductor structure 100 (i.e., providing the semiconductor structure 100 on a substrate that is different than the native growth substrate on which the semiconductor structure 100 was formed; also referred to herein as a non-native substrate). It will also be understood that it light sources according to embodiments of the present disclosure based on any of the microLED configurations (e.g., 20, 30a, 30b, 40a, 40b, 50, 60a, 60b, 70) as described herein may be included in large arrays (e.g., hundreds or thousands or many tens of thousands of the microLEDs), where the number of microLEDs in the array may be selected to realize the overall amount of desired light output from the light source.

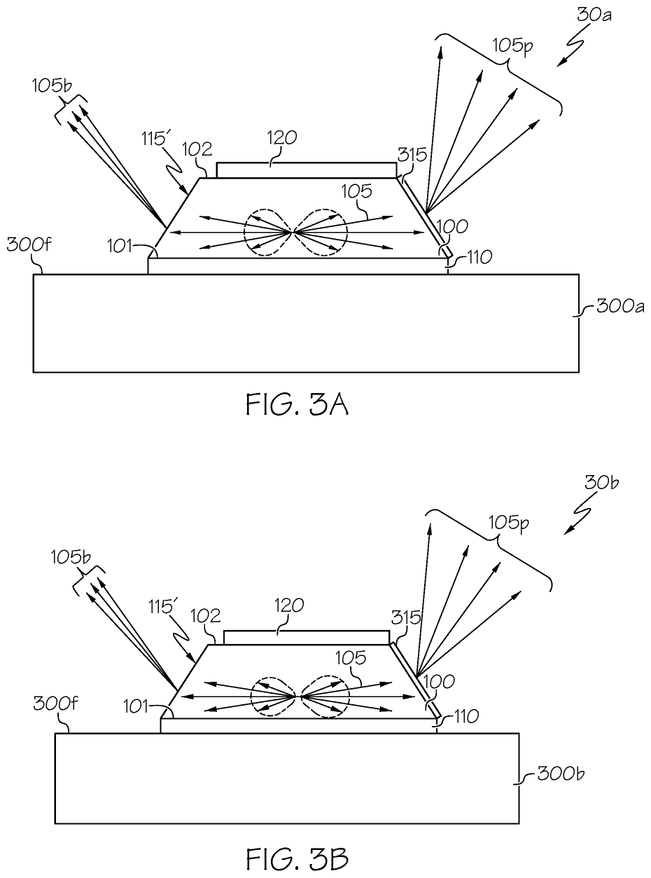

Further embodiments described herein illustrate example modification of some elements of the microLED device shown in FIGS. 2A and 2B to improve or change performance. For example, FIGS. 3A and 3B are schematic cross-sectional views illustrating configurations of microLED light sources 30a, 30b including sidewalls 115′ that are configured to direct the far-UVC light 105 in one or more desired directions according to some embodiments of the present disclosure.

In particular, in FIGS. 3A and 3B, the semiconductor structure 100 of the microLED 30a, 30b includes sidewalls or lateral surfaces 115′ that are inclined or angled (i.e., non-perpendicular with respect to the surfaces 101, 102 having the electrical contacts 110, 120 thereon). The sidewalls or lateral surfaces 115′ may be optimized or otherwise configured to direct the far-UVC light 105 upward out of the plane of the array (e.g., in a direction away from the native substrate 300a or non-native substrate 300b). In some embodiments, a respective sidewall 115 may include multiple angles of inclination (e.g., may be multi-faceted as it extends between the top and bottom surfaces of the semiconductor structure 100) in some embodiments. In other embodiments, the sidewalls may have more complex structuring in order to increase the output fraction of light or to modify the far field pattern angle distribution of the light that is output. Further examples of sidewall 115 modifications for emission of the far-UVC light 105 in one or more desired directions may be achieved by varying or altering the crystal orientation of the semiconductor material (e.g., by providing sidewalls 115′ that are offcut relative to one or more crystallographic axes of the semiconductor material) and/or the surface roughness of the sidewalls. The optical modifications to the sidewalls 115′ of the microLED may be optimized or otherwise configured to spread the light into a particular distribution pattern 105p (as shown in FIG. 3B), or to direct the light as or into a beam 105b (as shown in FIG. 3A), for example, by providing a coating or pattern 315 on the sidewalls 115′. More generally, one or more of the sidewalls 115′ of the semiconductor structure 100 may be configured to provide a light extraction surface that directs the far-UVC light 105 into a beam 105b or distributed light pattern 105p.

In some embodiments, the microLED 30a may be provided on and supported by the native substrate 300a (e.g., AlN, sapphire, GaN, or other substrate) that was used for epitaxial growth of the semiconductor material of the semiconductor structure 100, as shown in FIG. 3A. In other embodiments, the microLED 30b may be provided on and supported by a non-native substrate 300b, (e.g., a silicon substrate or other substrate) which is different from the material or substrate (e.g., a source wafer) on which the LED semiconductor material or structure 100 was grown or formed. Transferring the microLED from its native substrate 300a (e.g., source wafer) to a non-native support substrate 300b may be accomplished by microtransfer printing in some embodiments, but it will be understood that embodiments described herein may include various fabrication methods and may not be limited to any particular fabrication method.

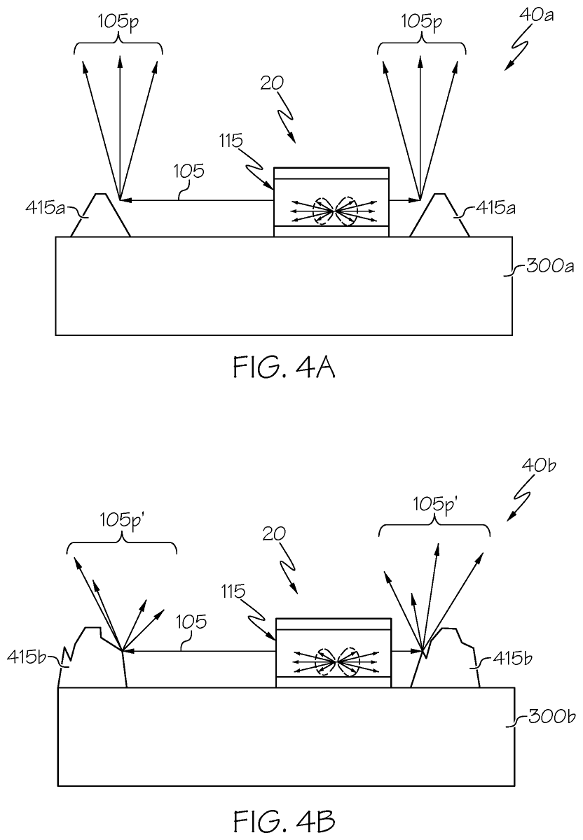

Still other embodiments of the present disclosure, such as those shown in FIGS. 4A and 4B, may include configurations in which the microLED 20, 30a, 30b is accompanied by reflective or other optical redirection structures 415 that are provided on or by the support substrate 300a, 300b. In particular, FIGS. 4A and 4B are schematic cross-sectional views illustrating configurations 40a, 40b of microLED light sources 20 on substrates 300a, 300b including optical redirection structures 415a, 415b (collectively 415) according to some embodiments of the present disclosure. While described hereinafter with reference to various configurations using the microLEDs 20 of FIGS. 2A and 2B, it will be understood that the illustrated microLEDs 20 may be replaced with other microLED configurations (e.g., microLEDs 30a, 30b with inclined sidewalls 115′ on native or non-native substrates 300a, 300b) in accordance with embodiments of the present disclosure.

The optical redirection structures 415 are arranged on a substrate 300a, 300b (e.g., attached to a front surface 300f of a non-native substrate adjacent the semiconductor structure 100) facing the light extraction surface 115 provided by the sidewall(s) of the semiconductor structure 100, and are configured to alter a propagation direction of the far-UVC light 105 that is output therefrom. For example, the optical redirection structures 415 may be reflective to the light of the far-UVC wavelength range, and may be shaped or otherwise configured to direct the far-UVC light 105 output from the lateral edges of the microLED into one or more desired directions or with a desired angular far field pattern.

FIG. 4A illustrates an example configuration 40a where the optical redirection structures 415a are patterned reflective structures (e.g., aluminum structures) that are fabricated with relatively planar or smooth angled edge surfaces that are configured to redirect the laterally-emitted light from the microLED into one or more different directions (shown as up and away from the support substrate 300a, 300b) to provide a controlled illumination pattern (e.g., a beam 105b or a desired distributed light pattern 105p). FIG. 4B illustrates an example configuration 40b where the optical reflection structures 415b are patterned reflective structures (e.g., aluminum structures) that include non-uniform or asymmetrical surfaces, so as to redirect the laterally-emitted light from the microLED into irregular or otherwise non-uniform illumination patterns in one or more direction(s) away from the support substrate 300a, 300b. The support substrate 300a, 300b may be a native substrate (e.g., a sapphire substrate for an AlGaN semiconductor structure) or a non-native substrate (e.g., a silicon substrate). In the examples 40a, 40b of FIGS. 4A and 4B, the optical redirection structures 415 are illustrated as being formed of different materials than the support substrate 300a, 300b and arranged on the surface of the support substrate 300a, 300b (e.g., using microtransfer printing or other techniques), but embodiments of the present disclosure are not so limited.

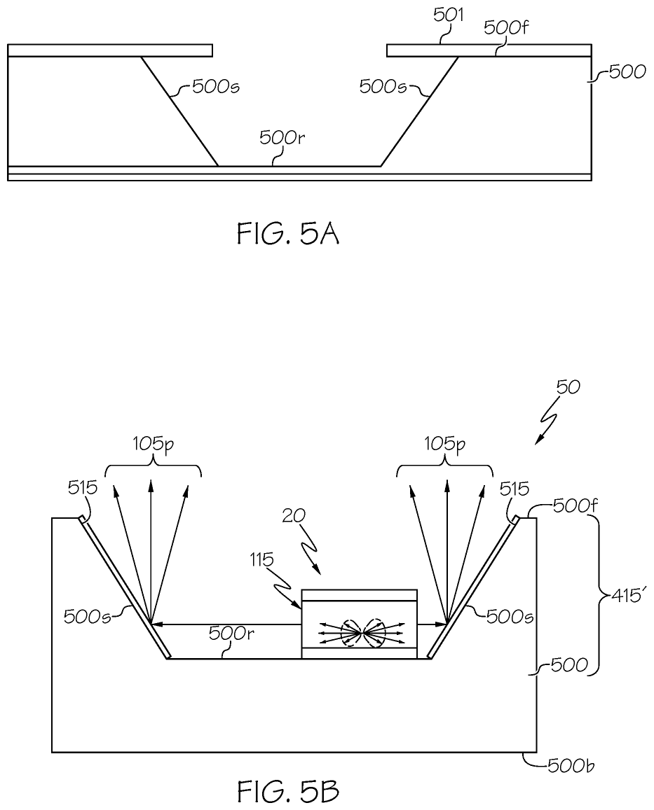

For example, further embodiments of the present disclosure may incorporate the optical or light redirection structures into the support substrate itself, such that additional features may not be required to be added onto the top surface of the support substrate. FIGS. 5A and 5B are schematic cross-sectional views illustrating configurations 50 of microLED light sources 20 on a substrate 500 including recessed surfaces 500r and adjacent side surfaces 500s that are configured to direct the far-UVC light 105 in one or more desired directions according to some embodiments of the present disclosure. The side surfaces 500s of the substrate 500 may thus provide optical redirection structures 415′ that are formed of the same material as and integral to the substrate 500, with the surface of the substrate 500 including the semiconductor structure 100 thereon recessed relative to the one or more optical redirection structures 415′.

For example, light redirection structures (illustrated in FIGS. 5A and 5B below by way of example with reference to inverted trapezoidal or “pyramid” structures defining recessed surfaces 500r or “pits” in the substrate 500) may be formed in or otherwise provided on the top surface of a non-native substrate 500 (e.g. a silicon or other support wafer), before placing or arranging the microLED device 20 on the recessed surface 500r inside the pit. In particular, the non-native substrate 500 may be patterned (e.g., by one or more selective or isotropic etch processes) to form the recessed surfaces 500r and surrounding side surfaces 500s, such that the side surfaces 500s define optical redirection structures 415′ that are configured to provide increased or improved light extraction.

As shown in FIGS. 5A and 5B, the side surfaces 500s of the inverted pyramid structures are formed at respective angles (relative to the recessed surface) to form optical redirection structures 415′ that are configured to direct the light that is emitted from the edge of the microLED 20 out of plane or to otherwise alter the propagation direction of the far-UVC light 105 away from the lateral direction. For example, the side surfaces 500s may be angled at about 45 degrees or less relative to the lateral direction of the light emission. The material(s) of the support substrate 500 and/or one or more layers thereon may be configured to be reflective to light of the far-UVC wavelength range. Some embodiments may further provide a reflection-enhancing coating or pattern 515 on the angled side surfaces 500s in order to enhance reflection of the far-UVC light 105 (e.g., with respect to efficiency and/or direction).

In particular, FIG. 5A illustrates that a mask 501 (e.g., an oxide or nitride mask) and an etch process (e.g., a TMAH or other isotropic etch process) may be used to selectively form one or more recessed surfaces 500r at a depth d relative to the top surface of a substrate 500 (e.g., a silicon substrate). The recessed surface 500r may have a width W_{bottom} that is narrower than the width Wₜₒₚ of the opening formed at the top surface of the substrate 500, and may extend along a {100} crystal plane or <100> crystal direction of the silicon substrate 500. The etch process may form side surfaces 500s in the silicon substrate 500 that are adjacent or surrounding the recessed surface, and extend along the {111} crystal plane or <111> crystal direction of the substrate 500m, at angles relative to the recessed surface 500r. The shapes of the side surfaces 500s are not limited to the examples shown in FIG. 5A, and more generally may include any features that may be etched or otherwise patterned into a support substrate 500 to define optical redirection structures 415′ facing the primary light extraction surfaces 115 of the microLED 20 and configured to alter a propagation direction of the far-UVC light 105 emitted therefrom into one or more directions.

FIG. 5B illustrates arrangement or placement of the microLED 20 on the recessed surface 500r of the substrate 500. The microLED 20 may be arranged centrally on the recessed surface 500r (i.e., at substantially equal distances to the respective side surfaces 500s), or may be offset toward one of the side surfaces 500s as shown. The process for arranging or placing the microLEDs on patterned surfaces of a substrate 500 may be, for example, microtransfer printing, but it will be understood that embodiments described herein are not limited to any particular fabrication method. Also, some embodiments may provide a single microLED on the recessed surface 500r inside each pit, while other embodiments may provide multiple microLEDs on the recessed surface 500r inside a respective pit (e.g., such that each recessed surface 500r includes one or more microLEDs thereon) with the light extraction surfaces 115 facing the optical redirection structures 415′ formed by the side surfaces 500s of the substrate 500.

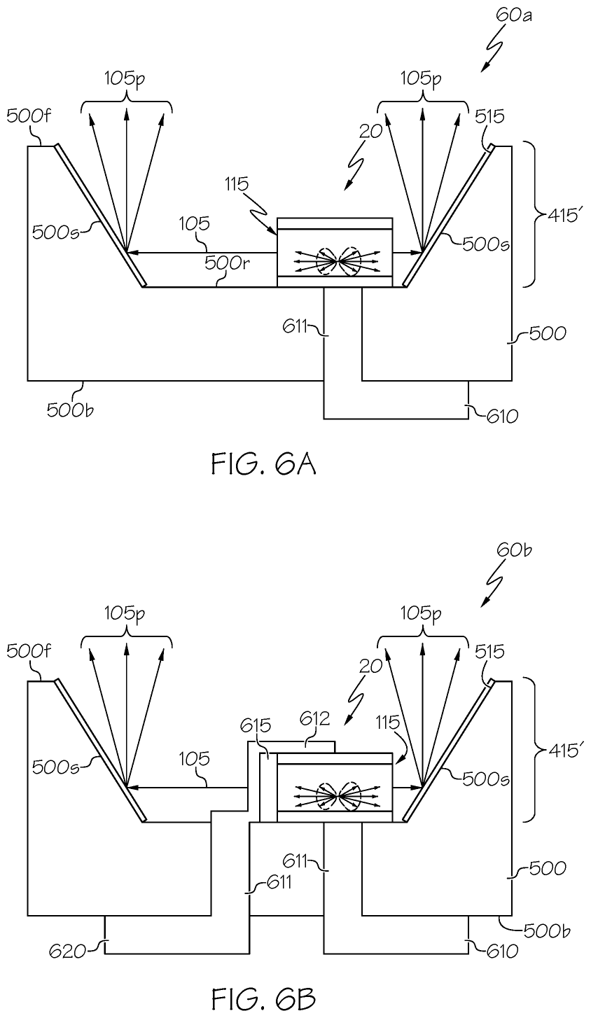

Yet further embodiments may provide one or more electrical connections to the electrical contacts 110, 120 of the microLEDs 20 by conductive through-vias 611 that extend through the support substrate 500. FIGS. 6A and 6B are schematic cross-sectional views illustrating configurations 60a, 60b of a microLED light source 20 on a substrate 500 including recessed surfaces 500r, side surfaces 500s that are configured to direct the far-UVC light 105 in one or more desired directions, and through-via 611 electrical connections extending therethrough according to some embodiments of the present disclosure.

As shown in FIGS. 6A and 6B, the substrate 500 includes a back surface 500b that is opposite the front surface 500f having the microLED 20 thereon. The back surface 500b includes one or more backside contacts 610, 620 thereon, which are electrically connected to the respective electrical contacts 110, 120 (e.g., the n-and p-contacts) of the microLED 20 by one or more through-wafer vias 611 (also referred to herein as conductive through vias 611) that extend through the substrate 500 and/or other conductive interconnections 612. FIG. 6A illustrates a configuration 60a including a single through via 611 per contact, which in this example contacts the n-type contact or N-terminal of the microLED. As shown in FIG. 6A, some embodiments may provide the microLED 20 directly on top of the through via. In other embodiments, as shown in FIG. 6B, multiple through-wafer vias 611 extend through the substrate 500 to provide electrical contact to both the N-and P-terminals of the microLED 20. As also shown in FIGS. 6A and 6B, an interlayer dielectric (ILD) 615 (or other non-conductive coating that is substantially transparent to the far-UVC light 105) may be provided to electrically isolate the sidewall 115 of the microLED 20 in order to prevent electrical shorting. Other embodiments may provide access and electrical connection to the top electrical contact (e.g., the p-contact) using other implementations, for example, wire bonding.

More generally, the through vias 611 may provide electrical connection between the front and back sides of the support substrate 500 having the microLED 20 thereon. In the examples 60a, 60b shown in FIGS. 6A and 6B, the through-vias 611 are implemented in combination with substrates 500 including the inverted pyramid or pit structures of FIGS. 5A and 5B, but it will be understood that through vias 611 may be used in combination with any of the substrates (e.g., 300a, 300b) described herein.

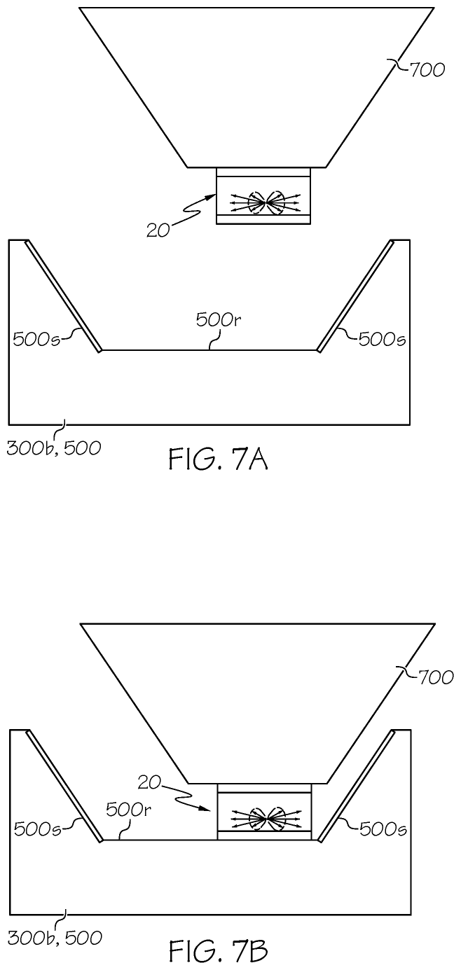

FIGS. 7A and 7B are schematic side views illustrating methods of fabricating a microLED light source 20 on a non-native substrate 500 according to some embodiments of the present disclosure. As shown in FIGS. 7A and 7B, in some embodiments, microtransfer printing techniques may be used as an assembly method for fabricating arrays (e.g., including hundreds or thousands) of microLEDs on a common support substrate 500. In microtransfer printing, the (microLED) devices may be removed from their native substrate (i.e., freed from native substrate on which the semiconductor structure 100 is formed) and placed or “printed” onto a different or non-native substrate 500 using an elastomeric or other stamp.

In particular, FIG. 7A illustrates that, prior to assembly, a non-native support substrate (e.g., 300b, 500) is provided. The non-native substrate 300b, 500 may include a front surface 300f, 500f that is configured to support one or more microLEDs, and a back surface opposite the front surface 300f, 500f. The front surface 300f may be a substantially planar surface, and in some embodiments, optical redirection structures 415′ may be arranged or placed on the front surface 300f of the substrate 300b between adjacent microLEDs 20 to redirect the propagation directions of far-UVC light 105 output therefrom into one or more directions away from the substrate. In other embodiments, as shown in FIG. 7A, the front surface 500f of the non-native substrate 500 may be a complex surface including a plurality of recessed surfaces 500r or pits surrounded by protruding side surfaces 500s that are configured to redirect the propagation directions of far-UVC light 105 output from one or more microLEDs 20 into one or more directions away from the substrate 500.

Still referring to FIG. 7A, a stamp may include respective posts that are sized to removably adhere one or more microLEDs 20 thereon. In some embodiments, the elastomeric transfer stamp may be configured and/or optimized in order to accomplish microtransfer printing of the microLEDs 20 onto complex or patterned substrates 500 such as described above. In particular, the elastomeric transfer stamp 700 may include posts with a height and/or tapered profile that provides sufficient clearance to extend between protruding side surfaces 500s of the substrate 500 surrounding a recessed surface 500r or pit therein, which may be different than the shapes of elastomeric stamps or posts that may typically be used for microtransfer printing onto smooth, flat substrates.

For example, a stamp post in accordance with some embodiments of the present disclosure may be shaped to (approximately) mirror or otherwise correspond to that of the inverted pyramid or other patterned or non-planar substrate geometry. The stamp configuration shown in FIGS. 7A and 7B may allow for an overall taller post that maintains sufficient strength, e.g., with a wider “base” (on the top) that is tapered towards the stamping surface so as to fit into the topology of the patterned or other non-planar substrate 500. As shown in FIG. 7B, the tapered posts of the stamp may fit into the dimensions of the patterned substrate 500 so as to transfer the microLED 20 from the post thereof to the recessed surface 500r of the substrate 500, in some instances directly onto the conductive through via 611 or other conductive interconnection on or exposed at the front surface 500f. As such, in some embodiments, the microLEDs may be transferred to a non-native support substrate 500 using elastomeric stamps whose posts are designed or otherwise configured to match or approximately correspond to the topology of the (complex) support substrate 500.

Transferring the microLEDs 20 to a non-native support substrate 500 as described herein (by microtransfer printing or other means) may be advantageous at least inasmuch that providing an array of multiple microLEDs 20 on their native substrate may be unmanageable. For example, the native substrate may typically be about 400 μm thick, while each microLED 20 may have respective lateral dimensions of about 10 μm, which may result in an undesirable aspect ratio and/or poor density or utilization of the substrate area. Moreover, there may be insufficient area on the top of each microLED 20 for a wire bond (which typically requires a 50 μm-75 μm wide metal bond pad). In contrast, microLEDs provided on a common non-native support substrate 300b, 500 as described herein may be connected by through vias 611 and/or other conductive interconnections 612 (including thin film interconnects) in and/or on the surface of the non-native substrate 300b, 500. The non-native substrate 300b, 500 may be optically transparent to far-UVC light in some embodiments.

According to some embodiments of the present disclosure, microLEDs (also referred to as μLEDs) with one or more lateral dimensions x, y of less than about 100 microns may provide higher optical efficiency at low (e.g. far UVC) wavelengths, which may result in higher power output based on or including (but not limited to) one or more of the following: higher light extraction efficiency; shorter distances to an edge or between opposing edges of the semiconductor material (i.e., higher perimeter to area ratio), on the order of the thickness t thereof; substantial or a majority of light extraction through the lateral surfaces 115 (sidewalls or edges) of the LEDs rather than through the top or bottom surfaces, which may allow for reduced (or no) transparency requirements for the electrical contacts 110, 120 on the top/bottom surfaces; and reduced electrical resistance of the electrical contacts 110, 120, due to the shorter lateral dimensions x, y of the surfaces 101, 102 of the epitaxial layer(s) of the semiconductor structure 100 on which the electrical contacts 110, 120 are provided.

In some embodiments, the microLEDs may have lateral dimensions of as small as 1 μm to as large as 20 μm (e.g., 1 μm×1 μm, 2 μm×2 μm, 3 μm×3 μm , 10 μm×10 μm, or 20 μm×20 μm), such that a perimeter-to-area ratio as high as about 4, or as low as about 0.2.

In some embodiments, the substrate (e.g., the native substrate) may be removed for light extraction through bottom or top surfaces of the semiconductor structure 100 (e.g., via flip chip techniques). In some embodiments, the top and/or bottom surfaces of the semiconductor structure 100 may be substantially free of light extraction features.

In some embodiments, a plurality of microLEDs as described herein may be arranged and electrically connected on a surface of a non-native substrate in an array configuration. The array may include a plurality of optical redirection structures on or otherwise protruding from the surface of the non-native substrate between respective ones of the microLEDs.

Embodiments of the present disclosure may thus address problems with light extraction efficiency of far-UVC light 105 generated by an LED (e.g., due to absorption by the p-contacts and/or by the semiconductor structure 100 itself) by recognizing that photons of shorter wavelengths may predominantly propagate at lateral angles (e.g., in lateral direction as show in the figures), and configuring the light emission or light extraction surface 115 of the LED to shorten the optical path length for output of the photons to increase or maximize output efficiency.

Some benefits of embodiments of the present disclosure may include, but are not limited to, improvement of performance of LEDs that operate with optical emission at wavelengths in the far-UVC wavelength range (about 200-240 nm). In particular LEDs that are fabricated using the Aluminum Gallium Nitride (AlGaN) material system may benefit, but benefits as described herein may also apply to other material systems and are not limited to GaN-based or Group III nitride based materials.

By improving the performance (i.e. optical output per unit electrical input) of LEDs in the far-UVC wavelength range, embodiments of the present disclosure may provide a new solid state source of light for use in germicidal UV applications, where presently the technology of AlGaN LEDs may be fundamentally challenged. Commercial applications for far-UVC illumination in accordance with embodiments of the present disclosure can include elimination of pathogens from air and/or surfaces in any indoor spaces where humans congregate (e.g., airports, schools, hospitals, inpatient care centers, workplaces, etc.), as well as in transportation vehicles (e.g., subway cars, trains, taxis, airplanes) and agricultural settings (e.g., animal production facilities, meatpacking facilities, indoor greenhouses, etc.).

Various embodiments have been described herein with reference to the accompanying drawings in which example embodiments are shown. These embodiments may, however, be embodied in different forms and should not be construed as limited to the embodiments set forth herein. Rather, these embodiments are provided so that this disclosure is thorough and complete and fully conveys the inventive concept to those skilled in the art. Various modifications to the example embodiments and the generic principles and features described herein will be readily apparent. In the drawings, the sizes and relative sizes of layers and regions are not shown to scale, and in some instances may be exaggerated for clarity.

The example embodiments are mainly described in terms of particular methods and devices provided in particular implementations. However, the methods and devices may operate effectively in other implementations. Phrases such as “example embodiment”, “one embodiment” and “another embodiment” may refer to the same or different embodiments as well as to multiple embodiments. The embodiments will be described with respect to systems and/or devices having certain components. However, the systems and/or devices may include fewer or additional components than those shown, and variations in the arrangement and type of the components may be made without departing from the scope of the inventive concepts.

The example embodiments will also be described in the context of particular methods having certain steps or operations. However, the methods and devices may operate effectively for other methods having different and/or additional steps/operations and steps/operations in different orders that are not inconsistent with the example embodiments. Thus, the present inventive concepts are not intended to be limited to the embodiments shown, but are to be accorded the widest scope consistent with the principles and features described herein.

It will be understood that when an element is referred to or illustrated as being “on,” “connected,” or “coupled” to another element, it can be directly on, connected, or coupled to the other element, or intervening elements may be present. In contrast, when an element is referred to as being “directly on,” “directly connected,” or “directly coupled” to another element, there are no intervening elements present.

It will also be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

The terminology used in the description herein is for the purpose of describing particular embodiments only and is not intended to be limiting of the invention. As used in the description and the appended claims, the singular forms “a”, “an” and “the” are intended to include the plural forms as well, unless the context clearly indicates otherwise.

It will also be understood that the term “and/or” as used herein refers to and encompasses any and all possible combinations of one or more of the associated listed items. It will be further understood that the terms “include,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the present disclosure are described herein with reference to illustrations that are schematic illustrations of idealized embodiments (and intermediate structures) of the invention. As such, variations from the shapes of the illustrations as a result, for example, of manufacturing techniques and/or tolerances, are to be expected. Thus, the regions illustrated in the figures are schematic in nature and their shapes are not intended to illustrate the actual shape of a region of a device and are not intended to limit the scope of the invention.

Unless otherwise defined, all terms used in disclosing embodiments, including technical and scientific terms, have the same meaning as commonly understood by one of ordinary skill in the art to which this invention belongs, and are not necessarily limited to the specific definitions known at the time of the present invention being described. Accordingly, these terms can include equivalent terms that are created after such time. It will be further understood that terms, such as those defined in commonly used dictionaries, should be interpreted as having a meaning that is consistent with their meaning in the present specification and in the context of the relevant art and will not be interpreted in an idealized or overly formal sense unless expressly so defined herein. All publications, patent applications, patents, and other references mentioned herein are incorporated by reference in their entireties.

Many different embodiments have been disclosed herein, in connection with the above description and the drawings. It will be understood that it would be unduly repetitious and obfuscating to literally describe and illustrate every combination and subcombination of these embodiments. Accordingly, the present specification, including the drawings, shall be construed to constitute a complete written description of all combinations and subcombinations of the embodiments of the present invention described herein, and of the manner and process of making and using them, and shall support claims to any such combination or subcombination.

Although the invention has been described herein with reference to various embodiments, it will be appreciated that further variations and modifications may be made within the scope and spirit of the principles of the invention. Although specific terms are employed, they are used in a generic and descriptive sense only and not for purposes of limitation, the scope of the invention being set forth in the following claims.

1. A light emitting diode (LED), comprising:
a semiconductor structure comprising at least one epitaxial layer that is configured to generate far-UVC light,
wherein one or more dimensions of the at least one epitaxial layer in a lateral direction are within an order of magnitude of a thickness of the at least one epitaxial layer in a vertical direction.

2. The LED of claim 1, wherein the semiconductor structure comprises:
first and second surfaces having respective electrical contacts thereon; and
at least one sidewall that extends between the first and second surfaces and is configured to emit the far-UVC light.

3. The LED of claim 2, wherein the at least one sidewall comprises opposing sidewalls of the at least one epitaxial layer that extend between the first and second surfaces, and the one or more dimensions comprise a distance between the opposing sidewalls.

4. The LED of claim 2, wherein the at least one sidewall is configured to direct the far-UVC light into a beam or into a distributed pattern.

5. The LED of claim 2, wherein the at least one sidewall is inclined between the first and second surfaces.

6. The LED of claim 2, wherein at least one of the respective electrical contacts is at least partially opaque to the far-UVC light.

7. The LED of claim 1, further comprising:
a substrate including the semiconductor structure on a front surface thereof, wherein the substrate is different than a native substrate on which the semiconductor structure is formed.

8. The LED of claim 7, wherein the substrate comprises one or more optical redirection structures facing the at least one sidewall of the semiconductor structure and configured to alter a propagation direction of the far-UVC light emitted therefrom into one or more directions.

9. The LED of claim 8, wherein the one or more optical redirection structures are attached to the front surface of the substrate adjacent the semiconductor structure.

10. The LED of claim 8, wherein the one or more optical redirection structures are integral to the substrate, and the surface of the substrate including the semiconductor structure thereon is recessed relative to the one or more optical redirection structures.

11. The LED of claim 8, wherein the substrate comprises:
a back surface opposite the front surface and having a backside contact thereon; and
at least one conductive through via that extends through the substrate and electrically connects at least one of the respective electrical contacts of the semiconductor structure to the backside contact.

12. The LED of claim 1, wherein the LED is free of a native substrate of the semiconductor structure.

13. The LED of claim 1, wherein the semiconductor structure is further configured to generate UVC light.

14. A light emitting diode (LED), comprising:
a semiconductor structure that is configured to generate far-UVC light, the semiconductor structure comprising first and second surfaces;
respective electrical contacts on at least one of the first and second surfaces; and
a primary light extraction surface comprising at least one sidewall of the semiconductor structure that extends between the first and second surfaces and is configured to emit the far-UVC light.

15. The LED of claim 14, wherein the at least one sidewall comprises opposing sidewalls of one or more epitaxial layers of the semiconductor structure that extend between the first and second surfaces, and a distance between the opposing sidewalls is less than about 100 microns.

16. The LED of claim 15, wherein the distance between the opposing sidewalls is less than about 50 μm.

17. The LED of claim 15, wherein a thickness of the one or more epitaxial layers of the semiconductor structure is about 10 μm or less.

18. The LED of claim 15, wherein the distance between the opposing sidewalls is within an order of magnitude of the thickness of the one or more epitaxial layers of the semiconductor structure.

14. The LED of claim 14, wherein the at least one sidewall is inclined between the first and second surfaces.

20. The LED of claim 14, wherein at least one of the respective electrical contacts is at least partially opaque to the far-UVC light.

21.-27. (canceled)

28. A light emitting diode (LED), comprising:
a semiconductor structure that is configured to generate light comprising a wavelength in a far-UVC wavelength range, the semiconductor structure comprising first and second surfaces having respective electrical contacts thereon and one or more lateral surfaces extending between the first and second surfaces,
wherein a collective surface area of the one or more lateral surfaces is within a factor of 10 of a respective surface area of the top or bottom surface.

29.-36. (canceled)
